(19) United States
(12) Patent Application Publication
(10) Pub. No.: US 2018/0036496 A1
(43) Pub. Date: Feb. 8, 2018

(54) Medication Delivery System

(71) Applicant: James Riviello, Ocean City, NJ (US)

(72) Inventor: James Riviello, Ocean City, NJ (US)

(21) Appl. No.: 15/226,218

(22) Filed: Aug. 2, 2016

(51) Int. Cl. A61M 11/04; A61M 15/06; A61M 15/00

(52) U.S. Cl. CPC A61M 11/042; A61M 15/0021; A61M 15/003; A61M 15/06; A61M 2205/50; A61M 2205/587; A61M 2205/8206

(57) ABSTRACT

A medication delivery system for delivering an inhalable medication includes an electronic cigarette that may be sucked. A capsule is provided. The capsule is infused with a fluid chronic obstructive pulmonary disease medication. The capsule is removably positioned within the electronic cigarette. Thus, the electronic cigarette delivers the fluid chronic obstructive pulmonary disease medication when the electronic cigarette is sucked.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIE THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98.

The disclosure and prior art relates to medication devices and more particularly pertains to a new medication device for delivering an inhalable medication.

### BRIEF SUMMARY OF THE INVENTION

An embodiment of the disclosure meets the needs presented above by generally comprising an electronic cigarette that may be sucked. A capsule is provided. The capsule is infused with a fluid Chronic Obstructive Pulmonary Disease (COPD) medication. The capsule is removably positioned within the electronic cigarette. Thus, the electronic cigarette delivers the fluid COPD medication when the electronic cigarette is sucked.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a medication delivery system according to an embodiment of the disclosure.

FIG. 2 is a right side view of an embodiment of the disclosure.

FIG. 3 is an exploded perspective view of an embodiment of the disclosure.

FIG. 4 is a front view of an embodiment of the disclosure.

FIG. 5 is a cross sectional view taken along line 5-5 of FIG. 2 of an embodiment of the disclosure.

### DETAILED DESCRIPTION OF THE INVENTION

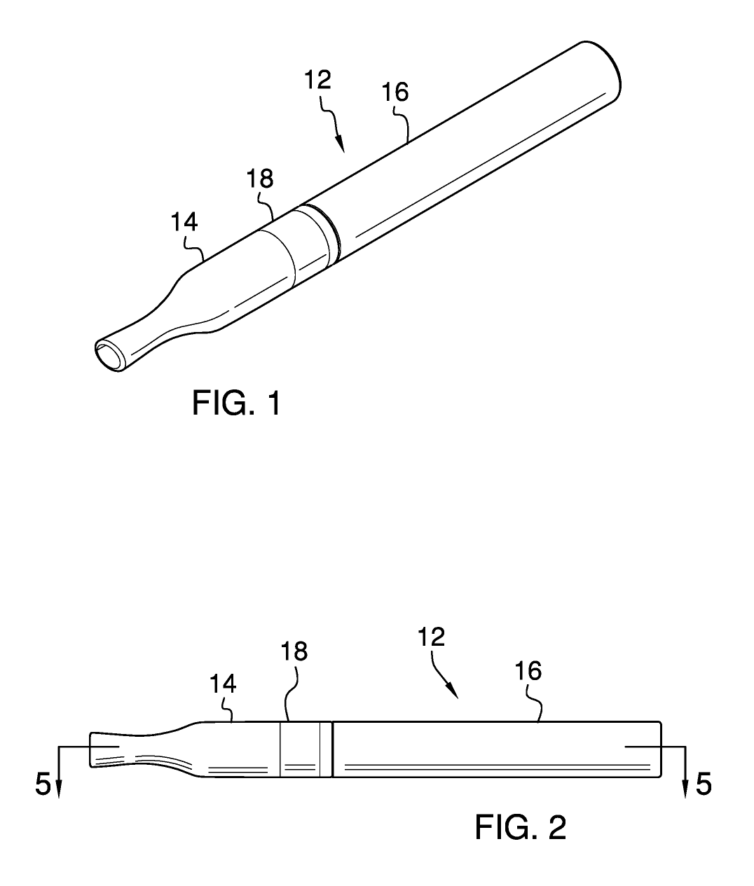

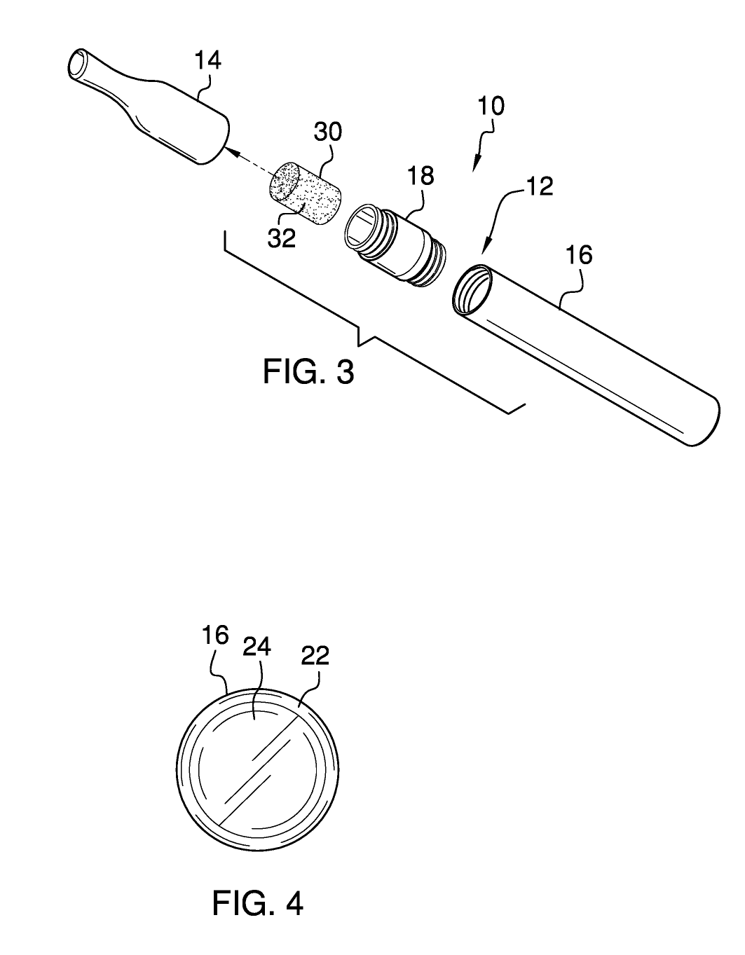

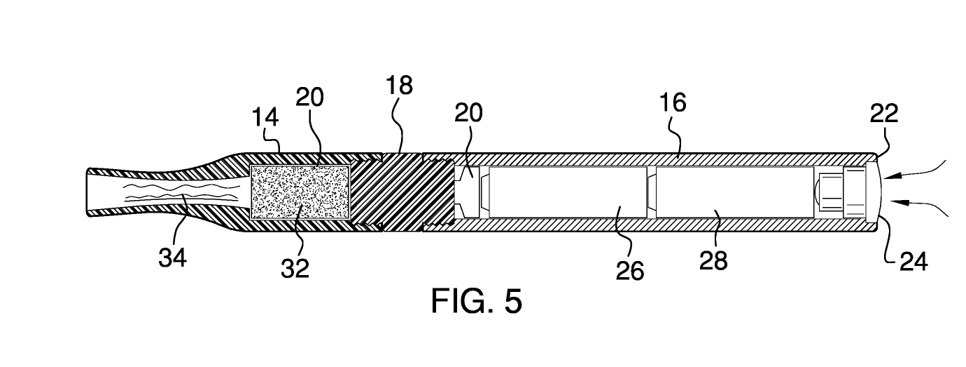

With reference now to the drawings, and in particular to FIGS. 1 through 5 thereof, a new medication device embodying the principles and concepts of an embodiment of the disclosure and generally designated by the reference numeral 10 will be described.

As best illustrated in FIGS. 1 through 5, the medication delivery system 10 generally comprises an electronic cigarette 12 that may be sucked. The electronic cigarette 12 includes a mouth piece 14, a tube 16 and a vaporizer 18. The vaporizer 18 is removably positioned between the tube 16 and the mouth piece 14. The mouth piece 14 includes a well 19 that is in fluid communication with the vaporizer 18. The electronic cigarette 12 may be an electronic cigarette 12 of any conventional design.

The electronic cigarette includes a processor 20. The processor 20 is positioned within the tube 16. The processor 20 detects when air is drawn through the tube 16. The processor 20 is electrically coupled to the vaporizer 18 when the vaporizer 18 is coupled to the tube 16. Thus, the processor 20 turns the vaporizer 18 on when air is drawn through the tube 16.

A tube 16 has a distal end 22 with respect to the vaporizer 18. A light emitter 24 is coupled to the distal end 22. A power supply 26 is positioned within the tube 16. The power supply 26 is electrically coupled between the processor 20 and the light emitter 24. Thus, the light emitter 24 is turned on when air is drawn through the tube 16. The power supply 26 may comprise at least one battery 28.

A capsule 30 is provided. The capsule 30 is comprised of a fluid permeable material. The capsule 30 is infused with a fluid chronic obstructive pulmonary disease (COPD) medication 24. The fluid COPD medication 32 may comprise tiotropium, salmeterol, formoterol, arformoteral, indacaterol, aclidinium of other long acting bronchodilator commonly used in a nebulizer for the treatment of COPD.

The capsule 30 is removably positioned within the electronic cigarette 12. Thus, the electronic cigarette 12 delivers the fluid COPD medication 32 when the electronic cigarette 12 is sucked. The capsule 30 is positioned within the well 19 such that the capsule 30 is in thermal communication with the vaporizer 18. The vaporizer 18 volatilizes the fluid COPD medication 32 when the electronic cigarette 12 is sucked. Thus, the volatilized fluid COPD medication 34 is inhaled without the use of a nebulizer or other inhaler. The combination of the fluid COPD medication 32 and the electronic cigarette 12 enhances privacy and efficiency of the treatment of COPD.

In use, the mouth piece 14 is removed from the electronic cigarette 12. The capsule 30 is positioned in the well 20 and the mouth piece 14 is coupled to the electronic cigarette 12. The mouth piece 14 is sucked when COPD symptoms are experienced. The vaporizer 18 volatilizes the fluid COPD medication 32 in the capsule 30 when the mouth piece 14 is sucked. Thus, the volatilized fluid COPD medication 34 is inhaled to relieve the symptoms of COPD. The electronic cigarette 12 replaces a nebulizer for the treatment of COPD symptoms. The electronic cigarette 12 is easily carried in a pocket or the like thereby enhancing efficiency of the treatment of COPD.

With respect to the above description then, it is to be realized that the optimum dimensional relationships for the parts of an embodiment enabled by the disclosure, to include variations in size, materials, shape, form, function and manner of operation, system and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A medication delivery system being configured to deliver an inhalable medication, said system comprising:
an electronic cigarette being configured to be sucked; and
a capsule being infused with a fluid chronic obstructive pulmonary disease medication, said capsule being removably positioned within said electronic cigarette wherein said electronic cigarette is configured to deliver said fluid chronic obstructive pulmonary disease medication when said electronic cigarette is sucked.

2. The system according to claim 1, wherein and said electronic cigarette includes a mouth piece, a tube and a vaporizer, said vaporizer being removably positioned between said tube and said mouthpiece, said mouth piece including a well being in fluid communication with said vaporizer.

3. The system according to claim 1, wherein:
said mouth piece has a well being in fluid communication with said vaporizer; and
said capsule is positioned within said well such that said capsule is in thermal communication with said vaporizer, said vaporizer volatilizing said fluid chronic obstructive pulmonary disease medication when said electronic cigarette is sucked thereby facilitating said volatilized fluid chronic obstructive pulmonary disease medication to be inhaled.

4. A medication delivery system being configured to deliver an inhalable medication, said system comprising:
an electronic cigarette being configured to be sucked, said electronic cigarette including a mouth piece, a tube and a vaporizer, said vaporizer being removably positioned between said tube and said mouthpiece, said mouth piece including a well being in fluid communication with said vaporizer; and
a capsule being infused with a fluid chronic obstructive pulmonary disease medication, said capsule being removably positioned within said electronic cigarette wherein said electronic cigarette is configured to deliver said fluid chronic obstructive pulmonary disease medication when said electronic cigarette is sucked, said capsule being positioned within said well such that said capsule is in thermal communication with said vaporizer, said vaporizer volatilizing said fluid chronic obstructive pulmonary disease medication when said electronic cigarette is sucked thereby facilitating said volatilized fluid chronic obstructive pulmonary disease medication to be inhaled.
